(19) United States
(12) Patent Application Publication
(10) Pub. No.: US 2026/0233033 A1
(43) Pub. Date: Aug. 13, 2026

(54) SYSTEM FOR REDUCING PATHOGENS IN A BREATHABLE AIRSTREAM

(71) Applicant: Thomas Dunbar, Dundee, NY (US)

(72) Inventor: Thomas Dunbar, Dundee, NY (US)

(21) Appl. No.: 19/537,452

(22) Filed: Feb. 11, 2026

Related U.S. Application Data
(60) Provisional application No. 63/757,023, filed on Feb. 11, 2025.

(51) Int. Cl. A62B 18/00; B01D 53/00; F21Y 115/10; F24F 8/22; F24F 8/30

(52) U.S. Cl. CPC A62B 18/003; B01D 53/007; F24F 8/22; F24F 8/30; A61L 2209/12; F21Y 2115/10

(57) ABSTRACT

A personal respiratory protection device includes a housing to be worn by a user. An airflow path extends through the housing from an air inlet to an air outlet. An ultraviolet irradiation assembly is in the airflow path and includes an array of ultraviolet emitters. A first ultraviolet-transmissive substrate is positioned close to the ultraviolet emitters. A diffuse reflective coating is on the first ultraviolet-transmissive substrate and defines apertures aligned with the ultraviolet emitters. A second diffuse reflective surface is spaced from the first ultraviolet-transmissive substrate to define a reflective irradiation cavity. A fan moves air through the reflective irradiation cavity and a control system is coupled to the ultraviolet light emitters and the fan. Air passing through the cavity is exposed to multiple diffuse reflections of ultraviolet radiation to produce an increased and spatially uniform photon density prior to delivery of treated air to the user.

### BACKGROUND OF THE INVENTION

### Field of the Invention

The present invention relates to pathogen reduction systems, and more particularly, to apparatuses and systems of reducing pathogens in an airstream passing through the pathogen reduction system, and still more particularly to a personal respiratory protection system and device for reducing pathogens in an airstream.

### Description of Related Art

Airborne transmission of pathogens and contaminants in enclosed environments presents ongoing challenges in healthcare, residential, commercial, and public spaces. Conventional approaches to air treatment typically rely on mechanical filtration, chemical treatment, or ultraviolet irradiation applied within HVAC ducts or standalone air purifiers. While ultraviolet irradiation systems may reduce airborne contaminant levels, they often suffer from limited photon utilization efficiency, non-uniform irradiation fields, or insufficient residence time for reliable inactivation of pathogens.

Previously disclosed systems for ultraviolet treatment of air in personal and handheld configurations included illumination chambers employing reflective surfaces to increase photon density. See U.S. Pat. Nos. 11,357,882 and 11,766,502, the entirety of each being hereby incorporated by reference. However, existing approaches generally rely on elongated chambers or point-source illumination geometries that do not efficiently scale to either compact wearable devices or large-area installations without significant losses in optical coupling efficiency, thermal management challenges, or uneven spatial distribution of ultraviolet energy.

Accordingly, there remains a need for ultraviolet air treatment systems that provide improved coupling of ultraviolet radiation into an airflow path, produce a substantially uniform and magnified photon density, and are adaptable across multiple form factors, including personal respiratory protection devices and room-scale or building-integrated installations. There is further a need for systems that coordinate ultraviolet emission with airflow and user breathing, and that integrate environmental sensing, thermal management, and building airflow architectures.

### BRIEF SUMMARY OF THE INVENTION

The present invention provides ultraviolet (UV) air treatment systems and methods employing a panel array of UV light emitters, also referred to herein as a flat flux capacitor/architecture. The flat flux capacitor/architecture enables efficient optical coupling, enhanced photon density, and scalable deployment from wearable personal devices to room-scale installations.

In one aspect, a personal respiratory protection device is provided in which an array of ultraviolet light emitters is positioned in close proximity to a first ultraviolet-transmissive substrate carrying a diffuse reflective coating with apertures aligned to the emitters. The apertures are sized larger than the emitting areas of the emitters such that a majority of emitted ultraviolet radiation is coupled into a reflective irradiation cavity formed between opposing diffuse reflective surfaces. Air is drawn through this cavity, where the ultraviolet radiation undergoes multiple diffuse reflections, producing an increased and spatially uniform photon density prior to delivery of treated air to a breathing zone of a user. Operation of the ultraviolet emitters may be synchronized with user inhalation, and the device may be coupled to wearable interfaces including eyewear frames and nasal delivery components.

In one configuration, the present disclosure provides a personal respiratory protection device that includes a housing configured to be worn by a user and having at least one air inlet and at least one air outlet. An airflow path extends through the housing from the at least one air inlet to the at least one air outlet. An ultraviolet irradiation assembly disposed in the airflow path, the ultraviolet irradiation assembly comprises an array of ultraviolet light emitters mounted on a circuit substrate; a first ultraviolet-transmissive substrate positioned in close proximity to the array of ultraviolet light emitters; a diffuse reflective coating disposed on the first ultraviolet-transmissive substrate and defining a plurality of apertures aligned with respective ones of the ultraviolet light emitters, each aperture having an area greater than an emitting area of its corresponding ultraviolet light emitter; and a second diffuse reflective surface spaced from the first ultraviolet-transmissive substrate to define, together with the first diffuse reflective coating, a reflective irradiation cavity. The plurality of apertures are configured to couple a majority of ultraviolet radiation generated by the ultraviolet light emitters into the reflective irradiation cavity while limiting direct line-of-sight emission into the airflow path. The device further includes at least one fan configured to move air through the reflective irradiation cavity and a control system operatively coupled to the ultraviolet light emitters and the at least one fan. Air passing through the reflective irradiation cavity is exposed to multiple diffuse reflections of ultraviolet radiation to produce an increased and spatially uniform photon density prior to delivery of treated air to a breathing zone of the user.

In a further configuration, the present disclosure provides a method of providing treated air to a user including a) drawing ambient air through a reflective irradiation cavity comprising opposing diffuse reflective surfaces and aperture-coupled ultraviolet light emitters; b) coupling ultraviolet radiation from the ultraviolet light emitters into the reflective irradiation cavity through apertures having areas greater than emitting areas of the ultraviolet light emitters; c) reflecting the ultraviolet radiation multiple times within the reflective irradiation cavity to increase photon density; and d) delivering the treated air to a breathing zone of the user in coordination with inhalation.

The present disclosure also includes an air treatment system for sanitizing air within an enclosed space that includes a wall-mounted or fixed housing configured for installation within a room or building. An airflow path extends through the housing and a scaled ultraviolet irradiation assembly is disposed in the airflow path. The scaled ultraviolet irradiation assembly comprises a large-area array of ultraviolet light emitters arranged in rows and columns on at least one circuit substrate; a first ultraviolet-transmissive substrate positioned in close proximity to the array of ultraviolet light emitters; a diffuse reflective coating disposed on the first ultraviolet-transmissive substrate and defining a plurality of apertures aligned with respective ultraviolet light emitters, each aperture having an area greater than an emitting area of its corresponding ultraviolet light emitter; and a second diffuse reflective surface spaced from the first ultraviolet-transmissive substrate to define, together with the first diffuse reflective coating, a reflective irradiation cavity extending over the large-area array. The plurality of apertures are configured to couple ultraviolet radiation from the large-area array into the reflective irradiation cavity while limiting direct line-of-sight emission into the airflow path. The system further includes at least one fan configured to draw air through the reflective irradiation cavity; a heat sink plate thermally coupled to the large-area array of ultraviolet light emitters; and a control unit configured to operate the ultraviolet light emitters and the at least one fan. Air flowing through the reflective irradiation cavity is exposed to multiple diffuse reflections of ultraviolet radiation to generate an increased and spatially uniform photon density for inactivation of airborne pathogens prior to discharge into the enclosed space.

In another aspect, a scaled air treatment system is provided for sanitizing air within rooms or buildings. A large-area array of ultraviolet light emitters is combined with a fused silica aperture substrate and an opposing diffuse reflective surface. The substrate and diffuse reflective surface form an extended reflective irradiation cavity spanning the emitter array. Air is drawn through the cavity by one or more fans, while a heat sink plate and auxiliary airflow channels provide thermal management of the emitters. The system may be wall-mounted or integrated with HVAC equipment such as mini-split heat exchangers, enabling sanitized air to be temperature-conditioned prior to discharge into an enclosed space.

In various embodiments, the scaled system further includes large-area filtration, controllable outside-air mixing, indoor air quality monitoring, and user interface displays.

Discharge airflow may be directed toward ceilings or architectural boundaries to promote room-scale circulation of sanitized air. Overlapping illumination patterns from adjacent emitters produce a substantially uniform optical field across the reflective irradiation cavity, enabling effective pathogen inactivation at throughput levels determined by emitter density, airflow rate, and optical coupling efficiency.

Collectively, these embodiments provide a modular ultraviolet air treatment platform in which the same flat flux capacitor principles are applied across personal and architectural scales, delivering enhanced photon utilization, uniform irradiation, and integrated airflow control for improved breathing and indoor air quality.

### BRIEF DESCRIPTION OF THE SEVERAL VIEWS OF THE DRAWING(S)

The accompanying drawings, which form a part of this specification, illustrate example embodiments of the invention. The drawings are not intended to be limiting, and like reference numerals generally refer to like elements throughout.

FIG. 1 is a perspective illustration of an exemplary embodiment of a wearable personal air treatment system shown on a user in accordance with an aspect of the present invention.

FIG. 2 is an exploded perspective view of the exemplary system shown of FIG. 1.

FIG. 3 is a side elevational view of the exemplary personal respiratory protection device shown in FIG. 2.

FIG. 4 is a top plan view of the exemplary personal respiratory protection device shown in FIG. 2.

FIG. 5 is an exploded view of the exemplary personal respiratory protection device shown in FIG. 2.

FIG. 6 is another exploded perspective view of the exemplary personal respiratory protection device shown in FIG. 2.

FIG. 7 is an exploded perspective view of an exemplary UV light assembly configured for use within the personal respiratory protection device shown in FIG. 2.

FIG. 8A is a front view of the exemplary personal respiratory protection device shown in FIG. 2.

FIG. 8B is an enlarged detail view of Detail A from FIG. 10A of the exemplary personal respiratory protection device shown in FIG. 2.

FIG. 9 is a side elevational view of an LED array circuit board assembly configured for use within the exemplary UV light assembly shown in FIG. 7.

FIG. 10 is a plan view of an example circuit board LED array shown in FIG. 9.

FIG. 11 is a side elevational view of an alternate LED array circuit board assembly configured for use within the exemplary UV light assembly shown in FIG. 7.

FIG. 12 is a plan view of a further mixed-wavelength LED array shown in FIG. 11.

FIG. 13 is a schematic sectional view illustrating optical angular extent from an exemplary LED emission surface of a single LED die of the exemplary UV light assembly shown in FIG. 7.

FIG. 14A is a perspective schematic view of a single LED die element of the exemplary UV light assembly shown in FIG. 7 illustrating a cone of ultraviolet light.

FIG. 14B is an enlarged detail view D of FIG. 14A.

FIG. 15 is an exploded view of a single LED die of the exemplary UV light assembly shown in FIG. 7.

FIG. 16 is a side schematic view illustrating multiple LED die elements arranged in a tiled configuration on a common LED circuit board with overlapping optical cones extending into a shared irradiation region.

FIG. 17 is an illustration of overlapping illumination patterns produced by adjacent LED die elements demonstrating generation of a substantially uniform optical field across the composite irradiation area.

FIG. 18 is a sectional view showing a stacked arrangement of the multiple LED die elements.

FIG. 19 is a sectional view similar to FIG. 18 further illustrating representative multiple diffuse reflection paths of ultraviolet radiation within the cavity.

FIG. 20 is a first cross-sectional view of the exemplary personal respiratory protection device illustrating an example airflow path during inhalation.

FIG. 21 is a second cross-sectional view of the exemplary personal respiratory protection device illustrating the cooling side airflow configuration system.

FIG. 22 is a front elevation of an exemplary wall-mounted architectural system in accordance with an aspect of the present invention.

FIG. 23 is a side view of the exemplary wall-mounted system of FIG. 27.

FIG. 24 is a perspective view of the exemplary wall-mounted system of FIG. 27.

FIG. 25 is an exploded perspective view of the exemplary wall-mounted system of FIG. 27.

FIG. 26 is an exploded schematic view of the large-area UV light assembly suitable for use within the exemplary wall-mounted system of FIG. 27.

FIG. 27 is a side sectional view of the large-area UV light assembly suitable for use within the exemplary wall-mounted system of FIG. 27.

FIG. 28 is a front plan view of the large-area UV light assembly suitable for use within the exemplary wall-mounted system of FIG. 27 showing a tiled LED array pattern.

### DETAILED DESCRIPTION

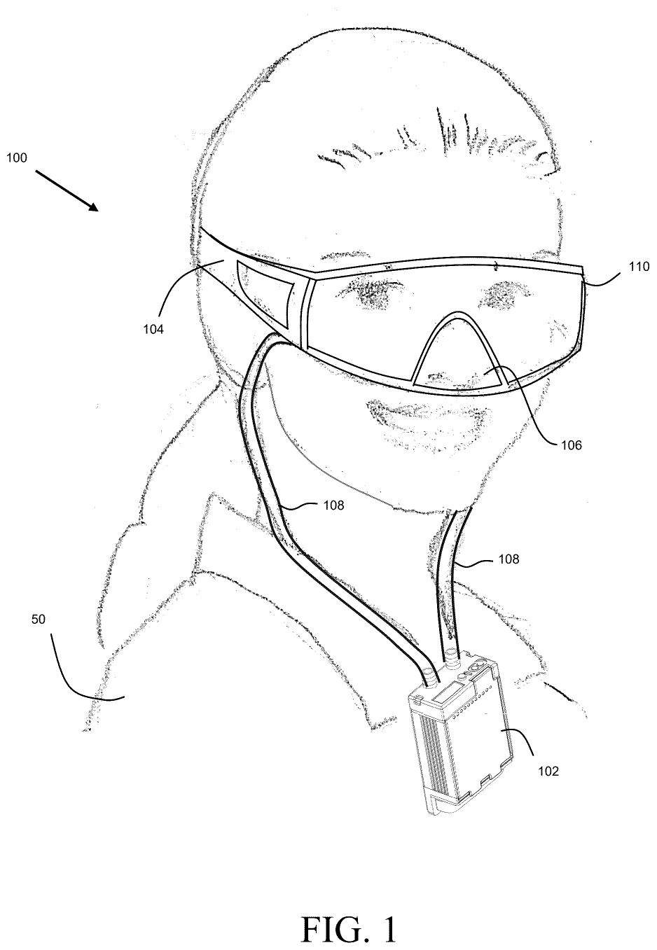

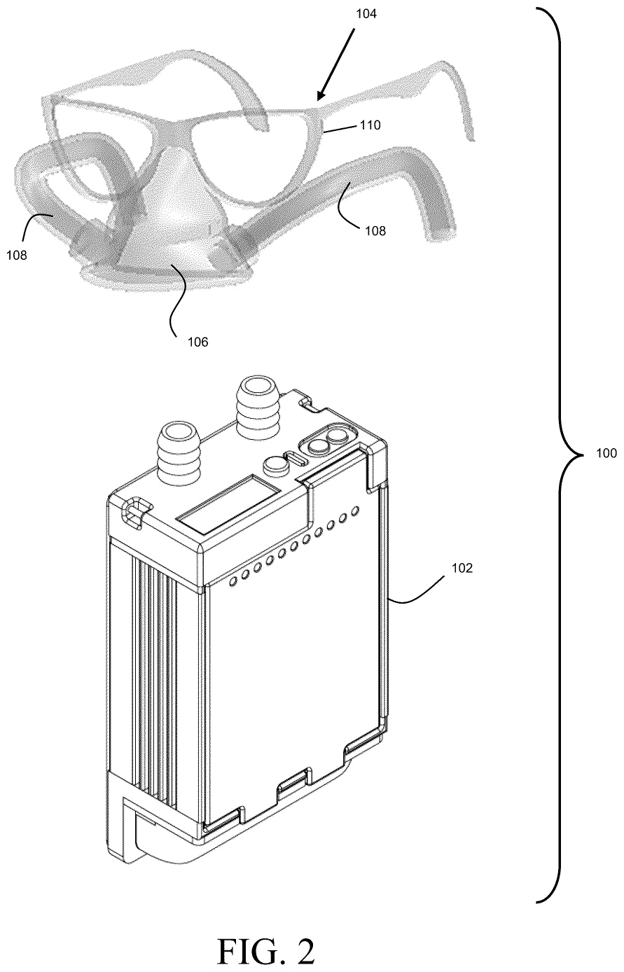

Turning now to FIGS. 1 and 2, an exemplary embodiment of a personal respiratory protection system 100 in accordance with the invention generally comprises a personal respiratory protection device 102 configured to be worn on a user 50, such as around the neck, and a wearable interface 104. The form factor of the personal respiratory protection device 102 is generally a device that can easily be worn about the body, such as but not limited being secured around a user's neck. Generally, it is preferred, although not necessary, that personal respiratory protection devices of the type disclosed herein have dimensions with a height between 3 and 12 inches, a width between 2 and 8 inches, and a depth between 1 and 4 inches.

As used herein, the terms personal respiratory protection system 100 and personal respiratory protection device 102 can encompass any system or device capable of inactivating pathogens in an airflow and embodiments of the present disclosure are not limited to the particular configuration of airflow pathogen reduction system/device 100/102. By pathogen, it is meant any virus, bacterium, or other disease-causing microorganism.

As shown in FIGS. 1 and 2, wearable interface 104 generally includes a nasal interface 106, such as a nasal mask or cannula configured to surround or otherwise engage the nose and/or mouth of user 50. Nasal interface 106 may be feed treated air from personal respiratory protection device 102 through one or more flexible air conduits or hoses 108. To assist mounting of nasal interface 106, as well as provide additional protection, if needed, wearable interface may further include an eyewear portion 110, such as but not limited to eyeglass frames (FIG. 2) or goggles (FIG. 1).

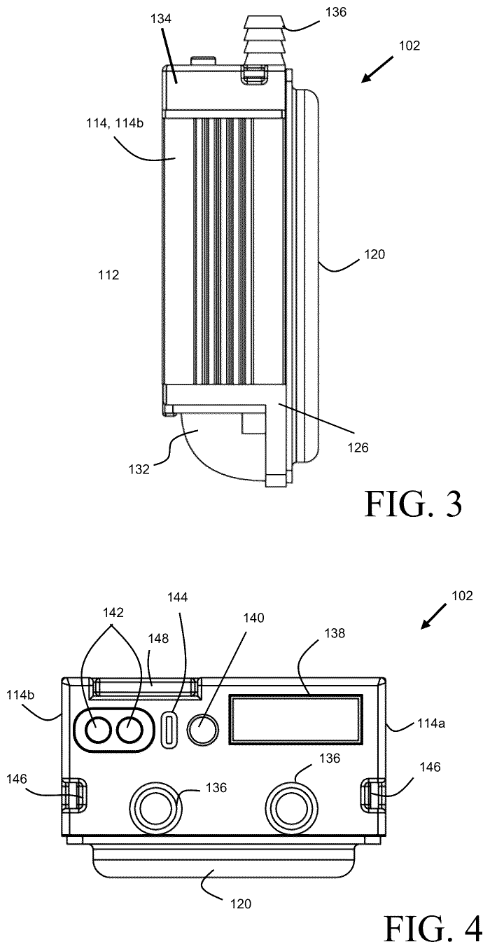

Turning now to FIGS. 3 and 4, personal respiratory protection device 102 generally includes a housing 112 comprising body 114; a front plate 116 which in combination with the body 114 define an electronics cavity 118, as will be described in greater detail below; a back plate 120 which in combination of body 114 define an air treatment chamber 122 configured to receive a ultraviolet (UV) light assembly 124 therein; a bottom cap 126 defining a plurality of air inlet apertures 128 configured to mountably receive a respective microfan 130 of a plurality of microfans; a lower airflow input unit 132 configured to mount to the bottom cap and envelope the microfans 130 and apertures 128 therein; and a top cap 134 which include one or more air outlets 136. As shown most clearly in FIG. 4, top cap 134 further includes user interface elements including a display 138 control buttons 140, 142, and a charging and communications port 144. Top cap 134 may also carry one or more latches 146, such at to receive a strap or lanyard such that personal respiratory protection device 102 may be worn about the neck of user 50 (FIG. 1).

Front plate 116 may be generally rectangular and a plurality of tabs that extend out from the rectangle shape in the same plane as front plate 116. By way of example and without limitation thereto, front plate 116 may include a top tab 148 and one or more bottom tabs 150.

Each of tabs 148,150 may contain an embedded magnet (not shown) configured to releasably magnetically couple front plate 116 to corresponding magnets embedded in top cap 134 and bottom cap 126. It should be understood that front plate 116 may alternatively or additionally be coupled to the device with other fasteners known in the art. Front plate 116 may also have a plurality of output apertures 116a that allow airflow to pass through front plate 116, from electronics cavity 118 to the ambient atmosphere, as will be described in greater detail below.

The apertures can vary in size and shape to allow different patterns and volumes of airflow to pass into the system. In an embodiment, a filter media (not shown) may sit between lower airflow input unit 132 and bottom cap 126 and may be any suitable filtration media known in the art for filtering an airflow, such as but not limited to, fiberglass, plastics, activated carbon, or plant-based filtration materials.

Front plate 116 and back plate 120 are configured to fit with body 114 and be framed by the top and bottom caps 126,134 to form generally airtight personal respiratory protection device 102. In one aspect of the invention, body 114 is a single piece which composes at least a portion of the external (i.e. side) walls 114a 114b of personal respiratory protection device 102. Body 114, in combination with front plate 116 and back plate 120, defines at least a portion of the internal spaces in personal respiratory protection device 102 with its shape, when personal respiratory protection device 102 is assembled. In an embodiment, body 114 is made from anodized aluminum material. In another embodiment, body 114 is made of a PTFE material such as POREX® Virtek™ PTFE made by Porex Filtration Group. In another embodiment, body 114 is made from another suitable material known in the art, such as metal, polymer, plastics, or another suitable material, and in exemplary embodiments may be constructed of a suitable heatsink material.

Body 114, in combination with front plate 116, defines electronics cavity 118 configured to receive electronics module 152. Electronics module 152 includes at least one controller 154 comprising electrical circuits, such as signal processors, and can be implemented as a programmed chip, as well as a dedicated processor or circuitry. The controller can be readily programmed to perform the recited calculations, or derivations thereof, to provide determinations of the detector as set forth herein. Controller 154 may also be communicatively coupled to an indoor air quality (IAQ) sensor unit 156.

Electronics module 152 comprises or is communicatively coupled to display 138, control buttons 140, 142, and charge port 144. Display 138 is configured to display information to a user. This can include battery status, filter life, light source life, date and time, and various other information that could be useful to a user. control buttons 140, 142 can communicate to the controller and control functions of the device or of the display 138. Charging port 144 provides a port for recharging batteries 158 using any of the power charging mating connectors known in the art. In one aspect of the present invention, recharging batteries 158 may comprise a stack of solid state battery panels. By way of example, recharging batteries 158 may comprise a ten layer stack of 3.6 V solid state batteries configured to output 36 V while being less than 5 mm thick.

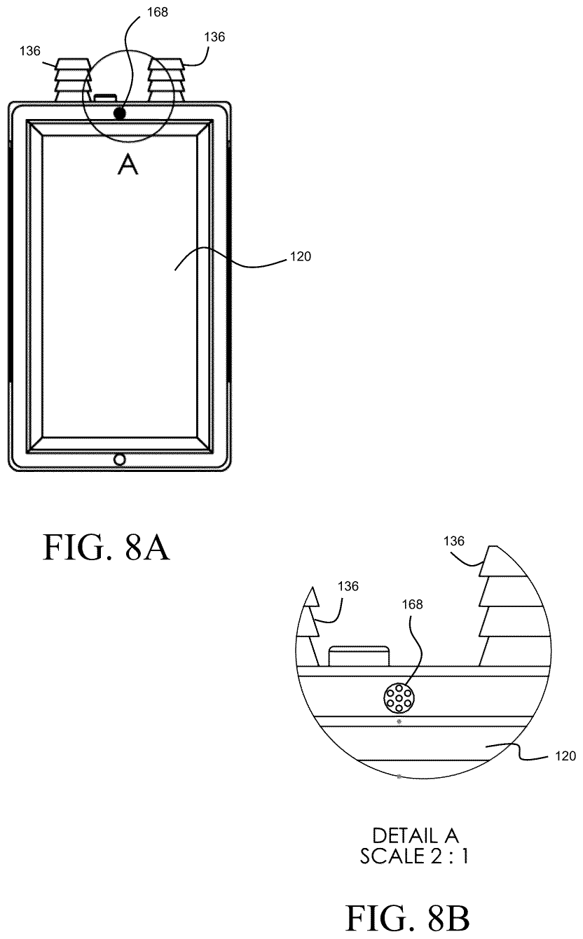

Body 114, in combination with back plate 120 defines, air treatment chamber 122 configured to receive UV light assembly 124. UV light assembly 124 generally comprises a stacked arrangement including an LED array substrate 160, diffuse reflective aperture coating 162 on a fused silica substrate cavity plate 164 defining a plurality of apertures 163, diffuse reflective coating 166 within back plate 120. As shown in FIGS. 8A, 8B, back plate 120 may further include an aperture 168 configured to allow sound to be recorded to an internal microphone (not shown) for system controller 154 to operate fans 130, such as when detecting an inhalation by user 50, as will be described in greater detail below. Alternatively, air treatment chamber 122 may be communicatively coupled to a pressure sensor under operational control of controller 154. The pressure sensor detects any change in pressure within air treatment chamber 122, thereby detecting for example if a user has inhaled from the device.

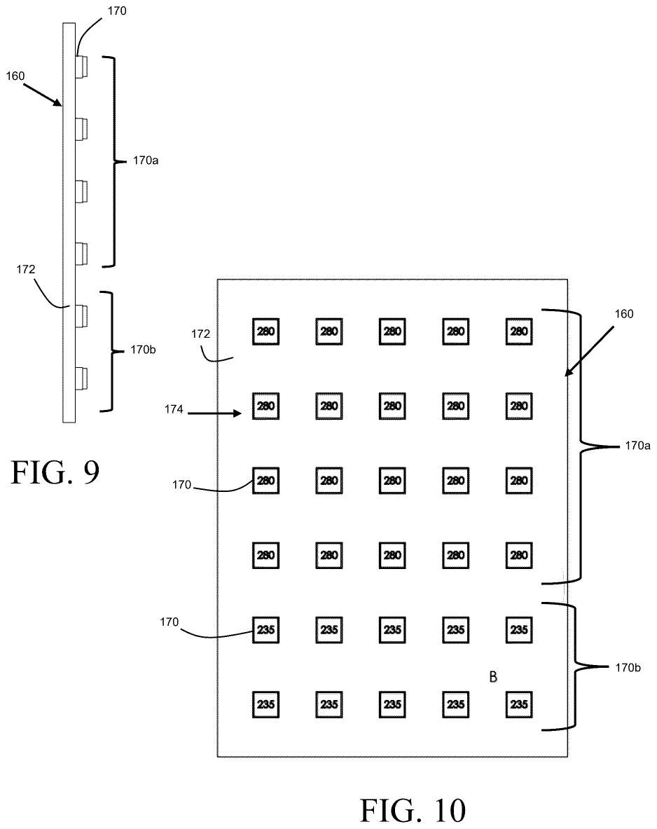

FIG. 9 is a side elevational view of an LED array circuit board assembly 160 showing relative placement of multiple LED die elements/LED emitters 170 along a circuit board 172.

FIG. 10 is a plan view of an exemplary circuit board LED array 174 illustrating grouped ultraviolet emitters of differing wavelengths, including emitters 170a operating to emit UV light having a wavelength of 280 nm and emitters 170b operating to emit UV light having a wavelength of 235 nm, arranged as an array across a circuit board 172.

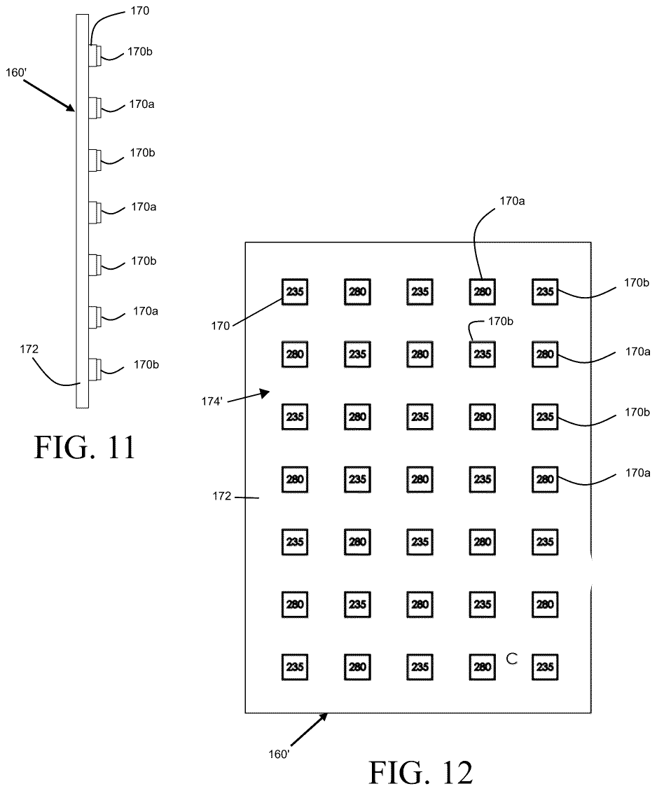

FIG. 11 is a side elevational view of an alternative LED array circuit board assembly 160′ showing relative placement of multiple LED die elements/LED emitters 170 along a circuit board 172. FIG. 12 is a plan view of an exemplary circuit board LED array 174′ illustrating an alternating array of ultraviolet emitters 170 of differing wavelengths, including emitters 170a operating to emit UV light having a wavelength of 280 nm and emitters 170b operating to emit UV light having a wavelength of 235 nm. It should be noted that while each LED array circuit board assembly 160 has been arranged as a five-by-six array of emitters, and size array may be employed depending on desired light density, available footprint, etc. It should be further noted that while only emitters of only two wavelengths have been shown and described, any number of emitters emitting any suitable wavelength(s) may be used in accordance with the invention.

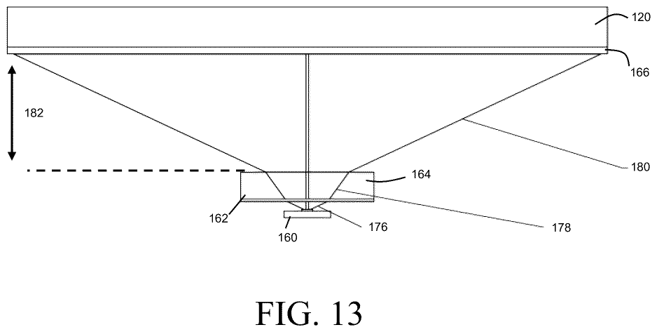

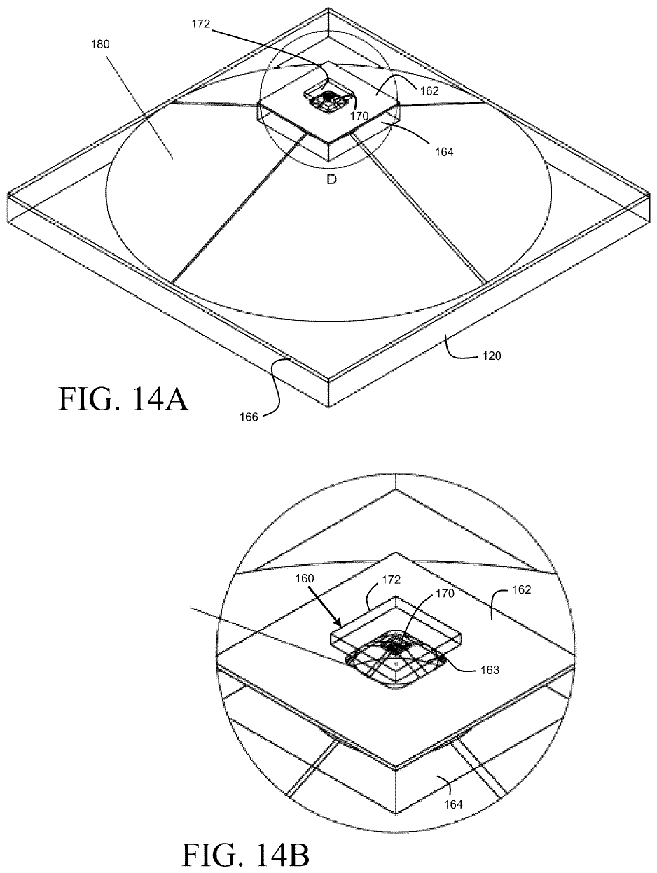

FIG. 13 is a schematic sectional view illustrating optical angular extent 176 from an LED emission surface 142 through the fused silica aperture substrate 137, 136 into the reflective irradiation cavity 182 (angular extent 180) defined by the opposing diffuse reflective surface 166 coated on the inner surface of second plate 120, including reduced angular extent 178 due to refraction within the substrate 136. FIG. 14A is a perspective schematic view of a single LED die element/LED emitter 170 illustrating a cone of ultraviolet light 180 propagating within the reflective cavity 182 above an LED element through reflective aperture 163 to opposite side reflective surface 166, 120. FIG. 14B is an enlarged view illustrating aperture 163 sizing and placement relative to the LED die element/LED emitter 170 such that a high percentage of light generated by the LED enters the reflective cavity 182 through diffuse aperture plate 162/aperture 163.

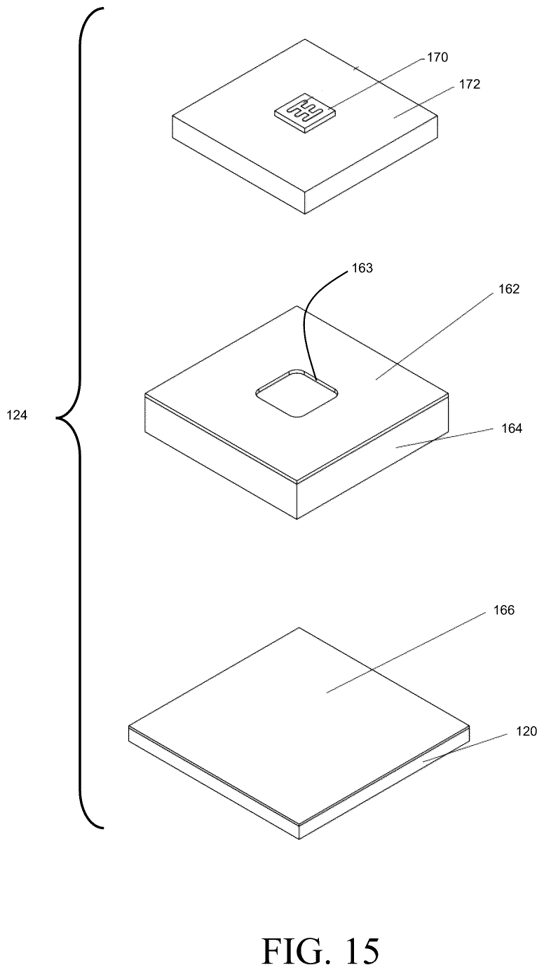

FIG. 15 is an exploded view of a single LED die element/LED emitter 170 within UV light assembly 124 including LED circuit board 142 with LED die element/LED emitter 170, fused silica aperture substrate 164 carrying highly reflective diffuse coating 162 with a clear aperture 163, and an opposing external substrate (e.g., back plate 120) with diffuse reflective coating 166, which altogether form reflective irradiation cavity 182.

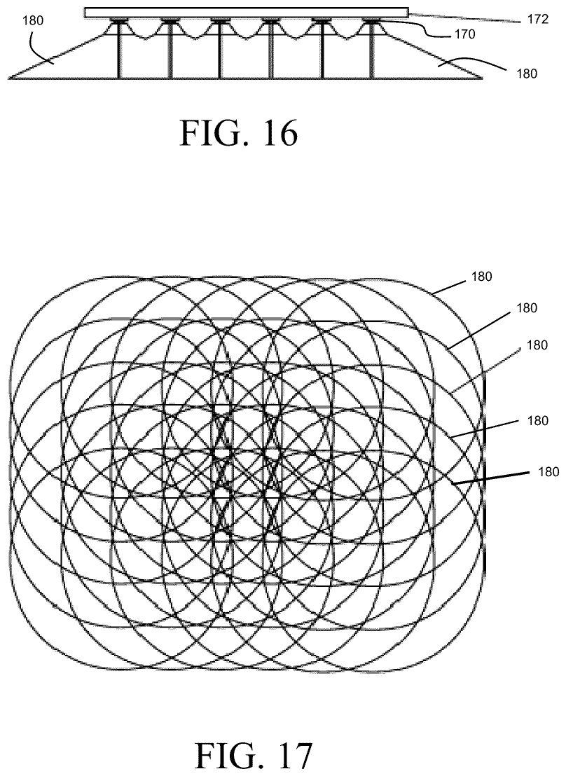

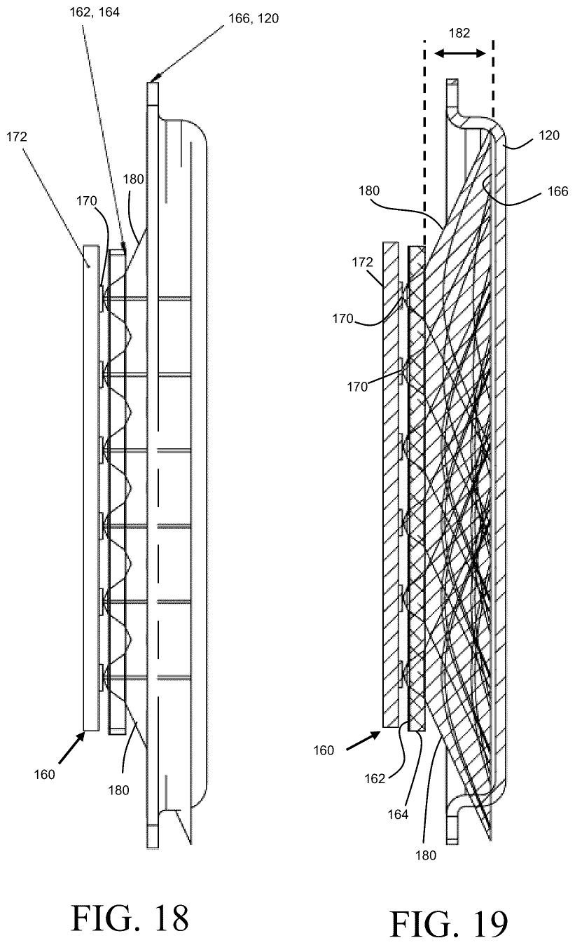

FIGS. 16-19 schematically illustrate multiple LED die elements/LED emitters 170 arranged in a tiled array configuration on a common LED circuit board 142. As shown, overlapping cones of ultraviolet light 180 extending into a shared irradiation cavity 182 so as to ensure total UV radiation coverage of irradiation cavity 182. It should be noted that this pattern is representative of one embodiment of a five-by-six array of LED die elements/LED emitters 170, and that other arrays may be employed with similar overlapping cones of irradiation. FIG. 18 is a cross-section view showing a stacked arrangement of LED die elements/LED emitters 170 on LED array circuit board 172, fused silica substrate 164 with aperture array 162, opposing diffuse reflective surface 166,120 while FIG. 19 is another cross-section view similar to FIG. 18 further illustrating representative multiple diffuse reflection paths 180 of ultraviolet radiation within the irradiation cavity 182.

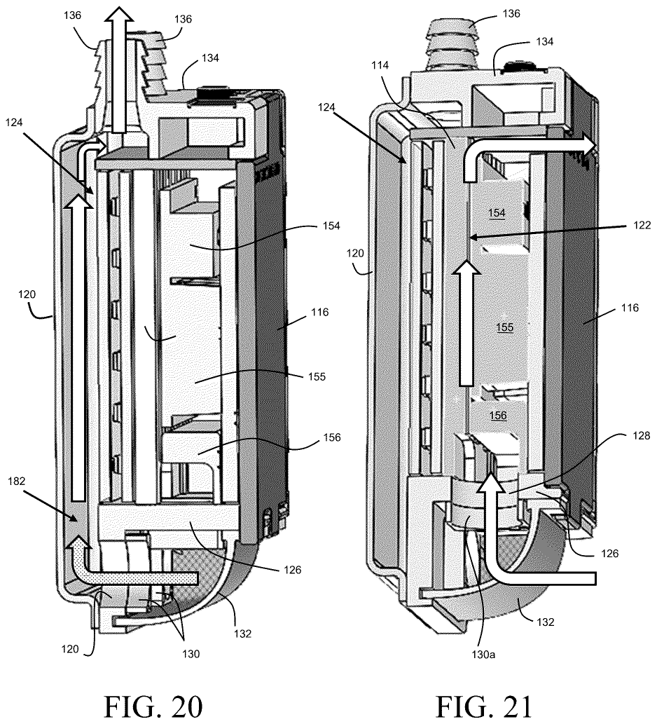

FIG. 20 is a cross-section view of personal respiratory protection device 102 illustrating an example airflow path during inhalation, including lower airflow input unit 132, reflective irradiation cavity 182, body 114, internal fan 130, control electronics module 154, LED driver modules 155 (see FIG. 6), air quality sampler module 156, and UI electronics module 138. Arrows indicate the flow of air through the “breathing” or air treatment side of personal respiratory protection device 102.

In normal operation of personal respiratory protection device 102, a user would inhale through one or more air outlets 136 (or an optional mouthpiece 136a; see FIG. 6). The inhale through air outlets 136 would move air from lower airflow input unit 132 through reflective irradiation cavity 182. Input of air may further be assisted using one or more microfans 130.

This inhalation may be detected by a microphone and/or a pressure sensor in communication with controller 156. The microphone/pressure sensor communicates with controller 156 providing a signal that the device 102 is in use, which would trigger LED driver modules 155 to switch on. UV-C exposure will inactivate pathogens in the air by the destruction of bonds within the pathogens' RNA, DNA or protein sheath. The reflective irradiation cavity 182 would then be actively eliminating pathogens in the airflow. The initiation timing, path length of the reflective irradiation cavity 182, the required time for generation of UV light and the microphone/pressure sensor sensitivity are configured to provide sufficient illumination of the passing air in the illumination chamber to provide the desired pathogen inactivation treatment. That is, once inhalation begins, the system must illuminate the passing air before a volume of the air has passed from the device to the user without having been illuminated.

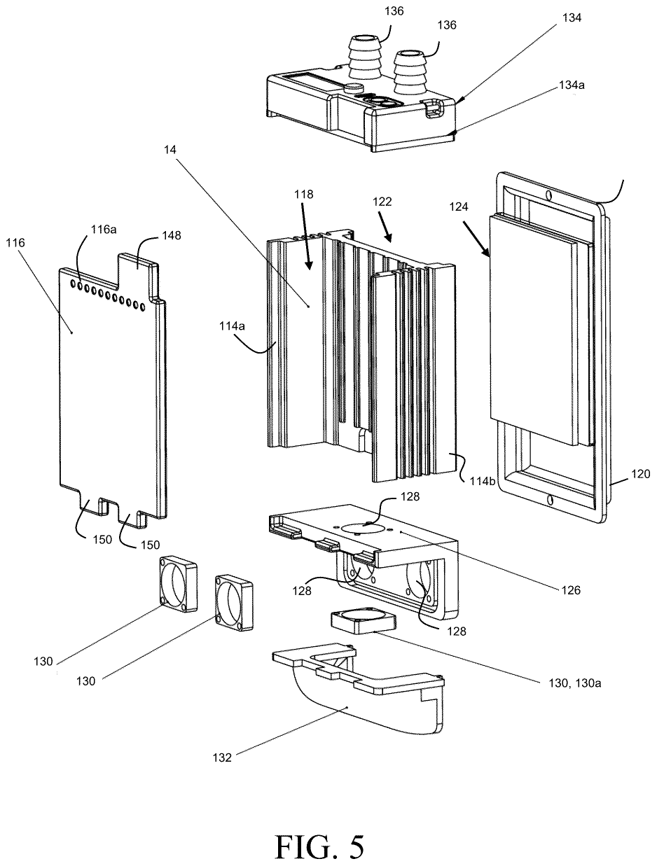

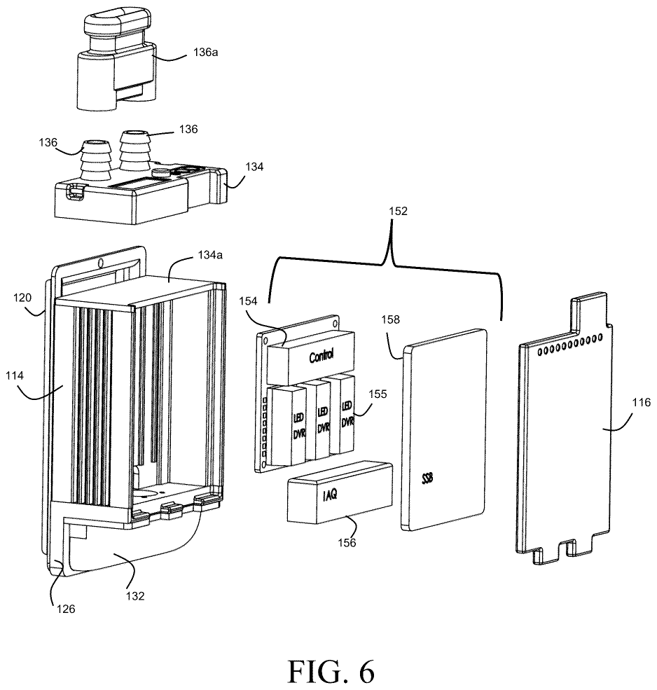

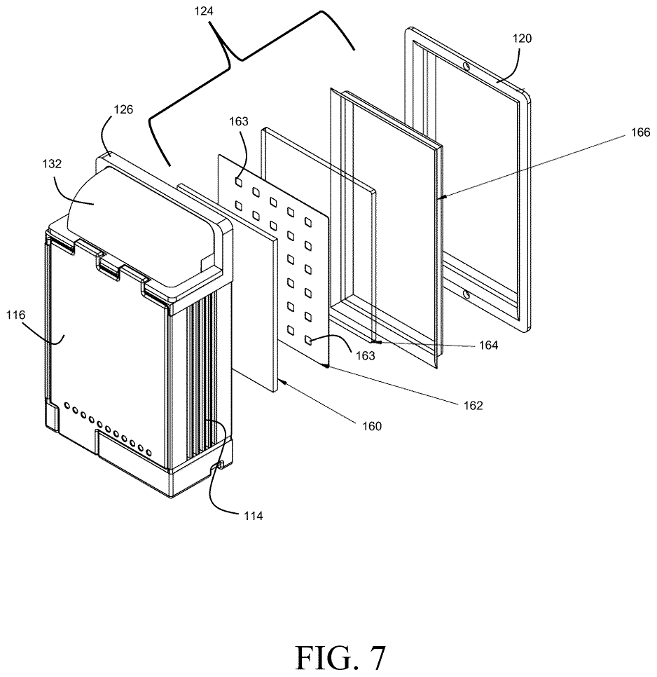

FIG. 21 is a cross-section view of the portable personal respiratory protection device 102 illustrating an exemplary flow path of air through the cooling side of device 102, i.e., electronics cavity 118 and inhalation cavity 122. When user 50 is not inhaling, cooling fan 130a (see FIG. 5) directs a flow of air into electronics cavity 118, if required, whereby control electronics module 156, LED driver modules 155, air quality sampler module 156, and UI electronics module 138, as well as LED die elements/LED emitters 170, may be cooled before the air exits front plate 116 through output apertures 116a.

Additionally, although the components of personal respiratory protection device 102 have been described in a unidirectional flow direction, in an embodiment the direction of the airflow is reversed. This embodiment allows a user who is the source of pathogens in the air (i.e. the user has contracted a virus and is actively shedding viral particles in their respiratory droplets and breath) to exhale into the air outlets 136 (or an optional mouthpiece 136a), having the exhaled breath trigger the microphone/pressure sensor, and treat the exhaled air with UV-C light before the air exhausts out the lower airflow input unit 132. This reverse flow embodiment would protect the surrounding people and ambient environment from exposure to the pathogens in the user's breath. This embodiment is advantageous where a user knows they are contagious, or where a user suspects they may be. It should be noted that reverse flow has been contemplated for all suitable embodiments for which the original airflow is described.

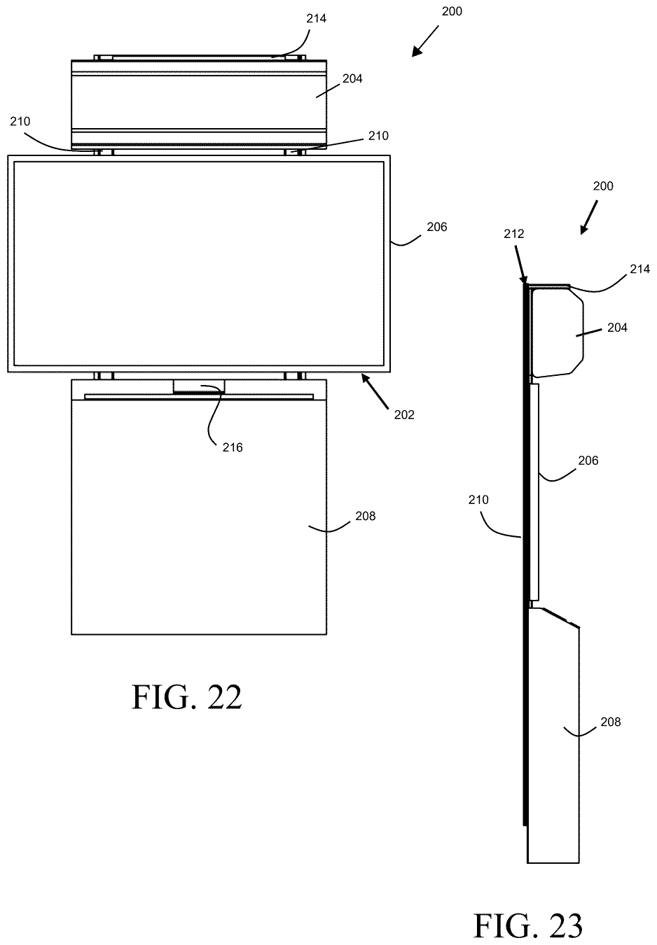

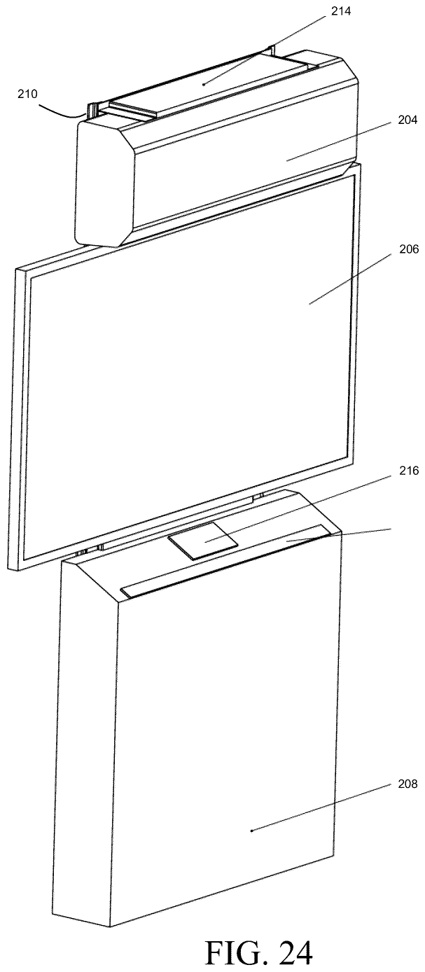

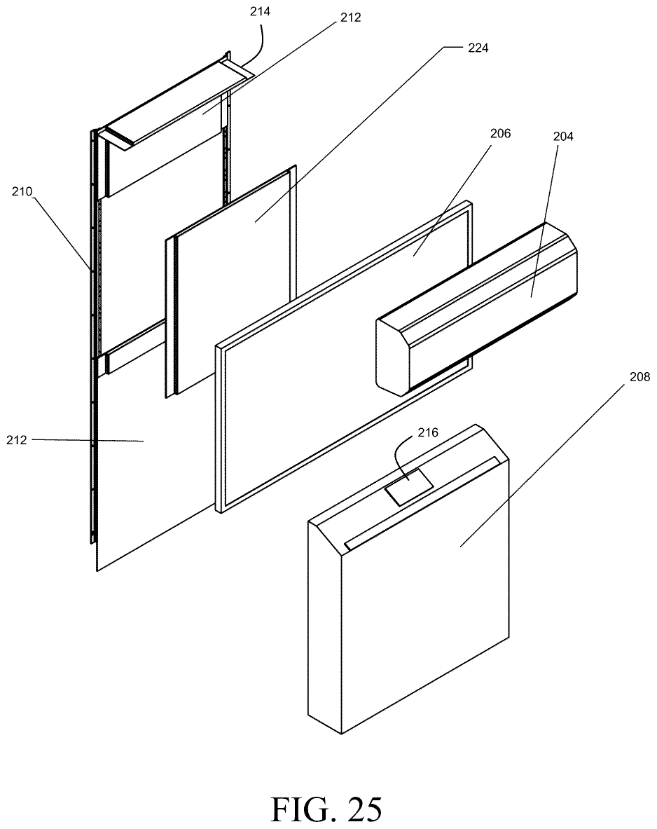

Turning now to FIGS. 22-25, shown is an exemplary embodiment of a wall-mounted architectural system 200 incorporating a scaled UV light assembly 224, a mini-split heat exchanger 204, a large screen display 206, and a lower fan/filter/system control unit 208. FIG. 23 is a side view of the wall-mounted system of FIG. 22 showing relative depth and vertical stacking of the mini-split heat exchanger 202, large screen display 203, and fan/filter/system control unit 204. Mounting surface 210 may be used to secure system 200 to a wall or other vertical member. An air flow cavity 212 may be located between units 204, 206, 208, 214, 224 and mounting surface 210 or the vertical surface.

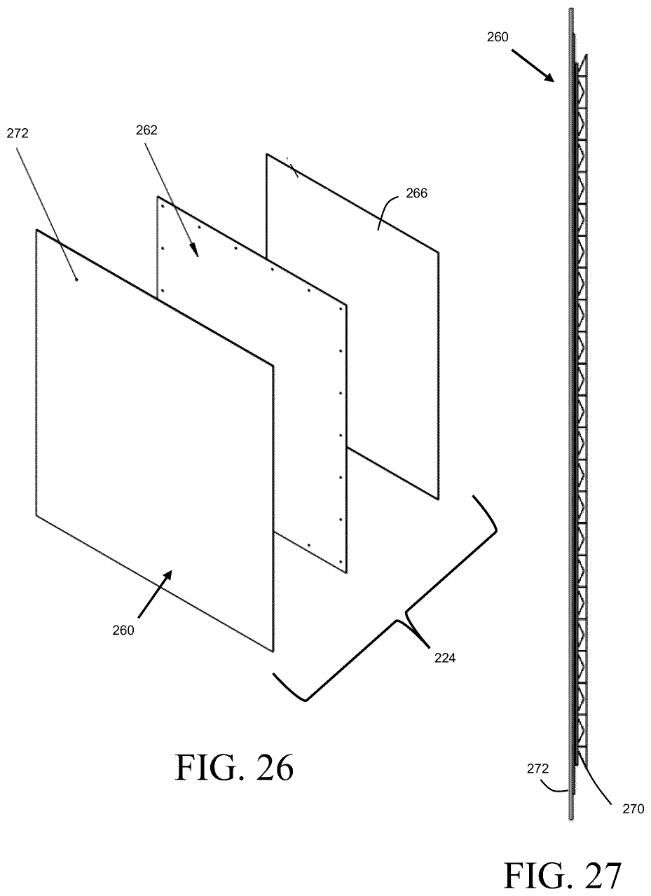

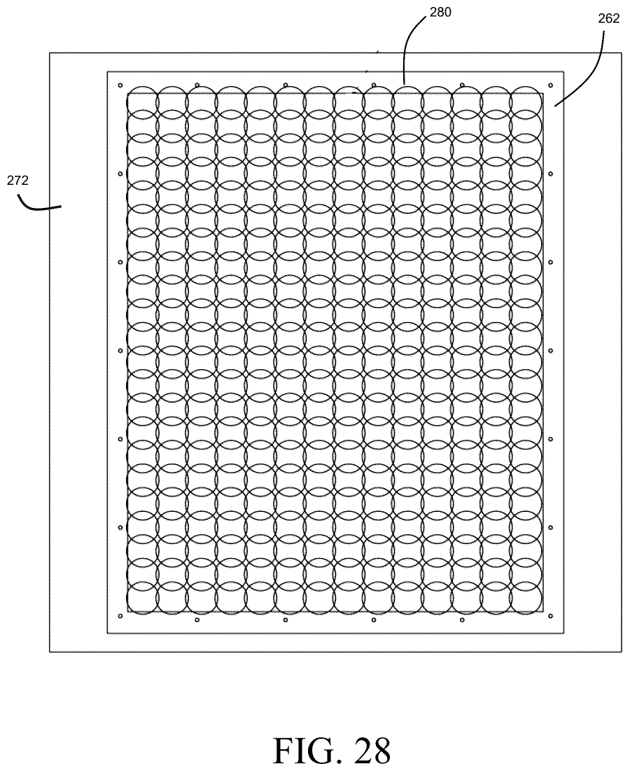

In use, ambient air is drawn into system 200 through fan/filter/system control unit 204 for upward passage through air flow cavity 212. Ambient air flows past UV light assembly 224 where UV-C exposure will inactivate pathogens in the air by the destruction of bonds within the pathogens' RNA, DNA, or protein sheath. UV light assembly 224 has a similar construction as UV light assembly 124 described above with regard to personal respiratory protection device 102 but has been scaled to treat a larger volume of air. By way of example and without limitation, as shown in FIGS. 26-28, UV light assembly 224 includes LED array substrate 260 comprising an array of LED die elements/LED emitters 170 on LED array circuit board 272, front diffuse reflective aperture plate 262 (aperture not shown for clarity), and a rear diffuse reflective plate 266. FIG. 27 illustrates the internal cavity depth and placement of the LED array relative to opposing diffuse reflective surfaces with LED emission pattern 280 shown for clarity of operation while FIG. 28 is a front plan view of the large-area UV light assembly 224 showing a tiled LED array pattern having emission pattern 280 defined by plates 272 and 266. By way of example and without limitation, UV light assembly 224 may comprise an array of fourteen (14)-by-twenty-two (22) LED die elements/LED emitters 170 on LED array circuit board 272, although other arrays may be contemplated depending upon air sanitation needs. After passing through UV light assembly 224, treated, sanitized air may exit system 200 through air output 214. Alternatively, if desired, treated, sanitized air may first pass through mini-split heat exchanger 204 for additional conditioning, e.g., temperature and/or humidity control.

Returning to FIGS. 22-25, large screen display 206 and/or control screen and IAQ monitor user interface 216 on lower fan/filter/system control unit 208 may be used to monitor and control operation of system 200. Filter access door 218 may provide access to an internal valve (not shown) for selecting outside air or recirculated indoor air within the lower system enclosure 208.

1. A personal respiratory protection device, comprising:
a) a housing configured to be worn by a user and having at least one air inlet and at least one air outlet;
b) an airflow path extending through the housing from the at least one air inlet to the at least one air outlet;
c) an ultraviolet irradiation assembly disposed in the airflow path, the ultraviolet irradiation assembly comprising:
i) an array of ultraviolet light emitters mounted on a circuit substrate;
ii) a first ultraviolet-transmissive substrate positioned in close proximity to the array of ultraviolet light emitters;
iii) a diffuse reflective coating disposed on the first ultraviolet-transmissive substrate and defining a plurality of apertures aligned with respective ones of the ultraviolet light emitters, each aperture having an area greater than an emitting area of its corresponding ultraviolet light emitter; and
iv) a second diffuse reflective surface spaced from the first ultraviolet-transmissive substrate to define, together with the first diffuse reflective coating, a reflective irradiation cavity,
wherein the plurality of apertures are configured to couple a majority of ultraviolet radiation generated by the ultraviolet light emitters into the reflective irradiation cavity while limiting direct line-of-sight emission into the airflow path;
d) at least one fan configured to move air through the reflective irradiation cavity; and
e) a control system operatively coupled to the ultraviolet light emitters and the at least one fan,
whereby air passing through the reflective irradiation cavity is exposed to multiple diffuse reflections of ultraviolet radiation to produce an increased and spatially uniform photon density prior to delivery of treated air to a breathing zone of the user.

2. The device of claim 1, wherein the ultraviolet light emitters comprise solid-state ultraviolet LEDs.

3. The device of claim 2, wherein the ultraviolet LEDs emit radiation at one or more wavelengths selected from approximately 222 nm, 235 nm, 254 nm, 265 nm, and 280 nm.

4. The device of claim 3, wherein different subsets of the ultraviolet LEDs emit different wavelengths to provide multiple pathogen inactivation mechanisms.

5. The device of claim 1, wherein the first ultraviolet-transmissive substrate comprises fused silica.

6. The device of claim 1, wherein the second diffuse reflective surface comprises a coated formed substrate positioned opposite the first ultraviolet-transmissive substrate.

7. The device of claim 1, wherein the reflective irradiation cavity defines a substantially planar or stacked geometry forming a flat flux capacitor.

8. The device of claim 1, wherein spacing between the first ultraviolet-transmissive substrate and the second diffuse reflective surface is selected to balance airflow residence time and photon density.

9. The device of claim 1, wherein the control system activates the ultraviolet light emitters only when airflow is detected through the reflective irradiation cavity.

10. The device of claim 9, further comprising a microphone or pressure sensor configured to detect a user breathing cycle, and wherein the control system synchronizes operation of the ultraviolet light emitters with inhalation of the user.

11. The device of claim 1, wherein the housing is coupled via tubing to a wearable interface selected from eyewear frames, nasal interfaces, or nose masks for delivery of treated air to the user.

12. The device of claim 11, wherein air channels are integrated into an eyewear frame and terminate at a silicone nasal interface.

13. The device of claim 1, further comprising an indoor air quality module configured to provide environmental or health monitoring feedback to the control system.

14. The device of claim 1, wherein the control system modulates fan speed to provide slight positive pressure during nasal inhalation.

15. The device of claim 1, wherein the reflective irradiation cavity is configured such that ultraviolet radiation undergoes multiple diffuse reflections, producing a photon density greater than a first-pass illumination level.

16. The device of claim 1, wherein the ultraviolet irradiation assembly is modular and removable from the housing.

17. The device of claim 1, wherein the device is supported on the user by a harness positioned near a collarbone region.

18. The device of claim 1, wherein the control system deactivates the ultraviolet light emitters during user exhalation.

19. The device of claim 1, wherein overlapping illumination patterns from adjacent ultraviolet light emitters produce a substantially uniform optical field within the reflective irradiation cavity.

20. A method of providing treated air to a user, comprising:
a) drawing ambient air through a reflective irradiation cavity comprising opposing diffuse reflective surfaces and aperture-coupled ultraviolet light emitters;
b) coupling ultraviolet radiation from the ultraviolet light emitters into the reflective irradiation cavity through apertures having areas greater than emitting areas of the ultraviolet light emitters;
c) reflecting the ultraviolet radiation multiple times within the reflective irradiation cavity to increase photon density; and
d) delivering the treated air to a breathing zone of the user in coordination with inhalation.

21-33. (canceled)
